(19) United States
(12) Patent Application Publication
(10) Pub. No.: US 2007/0095757 A1
(43) Pub. Date: May 3, 2007

(54) Methods and systems for the annotation of biomolecule patterns in chromatography/mass-spectrometry analysis

(76) Inventors: Anders Kaplan, Uppsala (SE); Lennart Bjorkesten, Uppsala (SE)

Correspondence Address: GE HEALTHCARE BIO-SCIENCES CORP.;PATENT DEPARTMENT, 800 CENTENNIAL AVENUE, PISCATAWAY, NJ, 08855, US

(21) Appl. No.: 10/560,728

(22) PCT Filed: Jul. 6, 2004
(86) PCT No.: PCT/EP04/07339

§ 371 Date: Dec. 14, 2005

(30) Foreign Application Priority Data
Jul. 21, 2003 (GB) 0316943.0

(51) Int. Cl. B01D 15/08

(52) U.S. Cl. 210/656; 210/198.2; 436/161; 436/171; 700/273

(57) ABSTRACT

The method and measurement system according to the invention performs combined Chromatography and Mass Spectrometry analysis and comprises the steps of: performing an C/MS analysis 300; generating at least one first elution profile 305, wherein one dimension is an elution time of the chromatography, and one dimension is mass to charge ratio (m/z), and at least one dimension a signal intensity, and the signal from each biomolecule species is dispersed forming a plurality of signal peaks associated with each biomolecule species in the elution profile; and reassembling 310 the dispersed signal originating from one biomolecule species in the elution profile. The reassembling comprises an automated annotation adapted to reassemble signal variations in the elution profile that originate from the same biomolecule species and generating a biomolecule map. The automated annotating is simultaneously based on both the elution time-dimension and the m/z-dimension.

### CROSS REFERENCE TO RELATED APPLICATIONS

This application is a filing under 35 U.S.C. § 371 and claims priority to international patent application number PCT/EP2004/007339 filed Jul. 6, 2004, published on Feb. 17, 2005 as WO 2005/015209, which claims priority to application number 0316943.0 filed in Great Britain on Jul. 21, 2003; the disclosure of which are incorporated herein by reference in their entireties.

### TECHNICAL FIELD OF INVENTION

The present invention relates to the study of biological samples containing a mixture of biomolecules, e.g. peptides, in order to identify, characterise and quantify individual biomolecules, and more particularly to methods and systems for profiling the relative abundance of at least some of the individual biomolecules across different experimental and biological conditions optionally defining a subset of biomolecules for identification or further characterisation.

### BACKGROUND OF THE INVENTION

A widespread method of studying protein content in biological samples is by using two-dimensional gel electrophoresis in combination with mass spectrometry, see for example, Kennedy, S., Toxicol. Lett. 2001, 120, 379-384. Two-dimensional gel electrophoresis is limited to the analysis of molecules with a molecular mass greater than approximately 10 kDa and there are no well-established methods to globally address the content of proteins and peptides below this limit.

Many of these smaller protein and peptide molecules play an important role in many biological processes and the advent of a method to routinely analyse peptide content in biological samples would therefore be a significant advance. Liquid chromatography (LC) coupled with mass spectrometry (MS) has emerged as a promising tool in proteomics capable of dealing with the inherent complexity in the biological samples and an increasing number of reports have been published illustrating the usefulness in combining LC and MS. It is suggested in “A neuroproteomic approach to targeting neuropeptides in the brain.”, Sköld K, Svensson M, Kaplan A, Björkesten L, Åström J and Andrén, Proteomics, 2, 447-454, that neuropeptides in the mass range of 300-5000 Da can be analysed by on-line nanoscale capillary reversed phase liquid chromatography (CRP LC) followed by electrospray ionisation quadrupole-time of flight mass spectrometry (Q-TOF MS). The article describes how the relative abundance of individual biomolecules across samples representing different experimental and biological conditions can be profiled and differences between the samples shown. Samples containing biomolecules were run through nanoscale CPR LC and Q-TOF MS. Each run resulted in an elution profile. Each individual data point in the elution profile represented an intensity value, or ion count, obtained from the MS detector for a particular chromatographic elution time and a particular m/z value. 3D representations of these elution profiles were drawn in which the y-axis showed the m/z ratio, the x-axis showed the elution time and the z-axis represented ion counts. Comparison between the different samples was performed by manually selecting similar regions on the 3 D representations of the different samples, integrating the ion counts within the regions and comparing the integrated ion counts of corresponding regions.

An LC/MS analysis can be pictured as a dispersion of the signal from each biomolecule species in the elution time and m/z dimensions and each peptide species will typically yield a plurality of peaks in the elution profile. If the resolution of the mass spectrometer is high enough, different isotopes of the same biomolecule species will be separated in the elution profile. Another type of dispersion of the signal is inflicted by the experimental method. In addition the biomolecules may receive different charge states during the experimental procedure. The different charge states will appear at different position in the elution profile. A further type of dispersion may arise from chemical pre-processing of the samples, for example mass labelling. In order to accurately compare relative abundances of biomolecule spices across different samples the dispersion of the signal originating from one peptide species has to be considered. In the method of Sköld et al, the different isotopes of one biomolecule species were manually identified and reassembled in an annotation process. The different charge states were not considered. Comparison between the 3D representations obtained from different samples was performed by manually selecting similar regions on the 3 D representations of the different samples, integrating the ion counts of the spots and comparing the integrated ion counts of corresponding regions. Since elution times of samples in LC columns may vary from run to run, it is not possible to simply overlay different representations of elution profiles on top of each other, instead the corresponding regions on the different representations have to manually identified, selected and marked so that they can be compared to each other.

Both the manual annotation and the manual process of finding corresponding regions in different elution profiles (samples) are extremely labour intensive and time consuming. The manual methods are not useful in large scale experiments or for industrial applications.

Several automated methods of processing LC/MS-data have been reported. In a number of methods, exemplified by “MoWeD, a computer program to rapidly deconvolute low resolution electrospray liquid chromatography/mass spectrometry runs to determine component molecular weights” by Pearcy and Lee, J am soc mass spectrum, 12, (2001) 599-606; and “Automated postprocessing of electrospray LC/MS data for profiling protein expression in bacteria.”, by Williams, Leopold and Musser, Anal chem 74, (2002) 5807-5813, individual mass spectra are deconvoluted by transformation methods. The methods offer an automated detection of peaks corresponding to peptides and are in some degree capable of handling the dispersed signals originating from the same peptide species. However, since only one or a few mass spectra are treated at the time and a transformation of the spectra is used, weak signals will often be ignored. In addition, the methods are noise sensitive as spurious noise peaks appearing in one or a few spectra, are easily mistaken as peaks originating from peptides. To reduce the effects of this problem hard filtration is used resulting in low sensitivity.

In “New algorithms for processing and peak detection in liquid chromatography/mass spectrometry data” by Hastings et al, Rapid comm mass spectrum 16, (2002) 462-467. a peak detection method is disclosed, “vectorized peak detection”, performed in a two dimensional representation, similar to the above described elution profiles. For a (elution time, m/z) position to be considered a peak, it must be a peak in the mass spectrum as well as a peak in the elution time dimension. The method is effective in avoiding spurious noise peaks, for example, but does not address the problem of dispersed signals.

The above mentioned studies illustrate the usefulness of LC/MS investigations. However, to make LC/MS-based analysis a method to be routinely used for analysing peptide content in biological samples further requirements have to be met. Most importantly, the method has to be able to screen a large amount of data and profile the relative abundance of some of the individual biomolecules across different experimental and biological conditions. In this the method has to address the problem of signal dispersion in the elution profiles. Due to the vast amount of data produced in a typical experiment, the method needs to be at least partly automated.

Furthermore, an attractive method needs to provide means for confirmation and validation of the result. This will be of special importance in fully automated methods and/or if advanced statistical methods like multivariate analysis are used, since these usually powerful analysis methods in certain cases can yield doubtful or misleading results even if the statistical measures indicate a high accuracy. In these cases an ability to compare the final results or an interim result with for example the unprocessed elution profiles would be of high value.

### SUMMARY OF THE INVENTION

The objective problem is to provide a method and measurement system of analysing LC/MS data for profiling the relative abundance of some of the individual biomolecules across different experimental and biological conditions adapted for the vast amount of data typically appearing in real experiments. Furthermore, it preferably should be possible to trace high level results back to their origins in the source data and it should be possible to define subsets of biomolecule species for further analysis.

The problem is solved by the method as defined in claim 1, the measurement system as defined in claim 19 and the computer program product defined in claim 23. Further improved methods and measurement systems have the features mentioned in the respective dependent claims.

The method of performing a combined Chromatography and Mass Spectrometry analysis (C/MS) according to the present invention comprises the steps of:
performing an C/MS analysis;
generating at least one first elution profile, which first elution profile is a multidimensional representations of the data resulting from the C/MS analysis wherein one dimension is an elution time of the chromatography, and one dimension is mass to charge ratio (m/z), and at least one dimension a signal intensity. The elution profile has a characteristic variation in the signal intensity which is an indication of the existence of a specific biomolecule species. The signal from each biomolecule species is dispersed forming a plurality of signal peaks associated with each biomolecule species in the elution profile; and
reassembling the dispersed signal originating from one biomolecule species in the elution profile. The reassembling step comprises an automated annotation adapted to reassemble signal variations in the elution profile that originate from the same biomolecule species and generating a biomolecule map. The automated annotating is simultaneously based on at least both the elution time-dimension and the m/z-dimension.

In one embodiment of the method according to the present invention the dispersion of signal from each biomolecule species arises from the existence of different isotopes and/or charge states of the biomolecule species, and the automated annotation reassembles, for essentially each biomolecule species, the signal dispersion caused by both the different isotopes and/or different charge states of the biomolecule species.

In another embodiment the sample comprises biomolecules species that have received different chemical labels, giving at least a first chemically labelled biomolecule with a first label and a second mass-labelled biomolecule with a second label. The chemical difference causes a further dispersion of the signal in the elution profile, and the automated annotation reassembles the signal dispersion caused by the chemical labelling.

In a further embodiment the automated annotation uses knowledge of the mass spectrometer resolution in the reassembling of dispersed signals.

In a still further embodiment of the present invention the automated annotation in the reassembling of dispersed signals uses a priori assumptions on the relations between different charge states and/or different isotopes of the same biomolecule species in the reassembling of dispersed signals. Alternatively, or in combination, the automated annotation uses resemblances detected during the analysis, for example in the signal pattern between different charge states, in the reassembling process.

One advantage afforded by the present invention is that the automated alignment makes it possible to screen a large amount of data and profile the relative abundance of some biomolecule species across different samples.

A further advantage is that the enhancement in the signal intensity afforded by the consensus profile can be used to detect weak signals typically corresponding to biomolecule species with low abundance.

Another advantage is that in the method according to the present invention it is possible to trace a high level result back to its origins in the source data, and to define subsets of biomolecule species for further analysis.

### BRIEF DESCRIPTION OF THE FIGURES

The features and advantages of the present invention outlined above are described more fully below in the detailed description in conjunction with the drawings where like reference numerals refer to like elements throughout, in which:

FIG. 1 is a schematic block diagram illustrating a system to practice the method of the invention;

FIG. 2a is an example of an elution profile produced by the system of FIG. 1;

FIG. 2b and FIG. 2c illustrate the signal dispersion caused by different isotopes and charge states;

FIG. 3a is a flowchart illustrating the main steps of the method according to the invention;

FIG. 3b is a flowchart illustrating the details of the annotating algorithm of the method according to the invention;

FIG. 4 shows schematically the usefulness of the method according to the present invention in comparison with prior art methods;

FIG. 5 shows schematically the elution profiles of an 2DLC/MS experiment; and

FIG. 6 shows how the method according to the invention is used to reassemble different chemical labels in an elution profile.

### DETAILED DESCRIPTION OF THE INVENTION

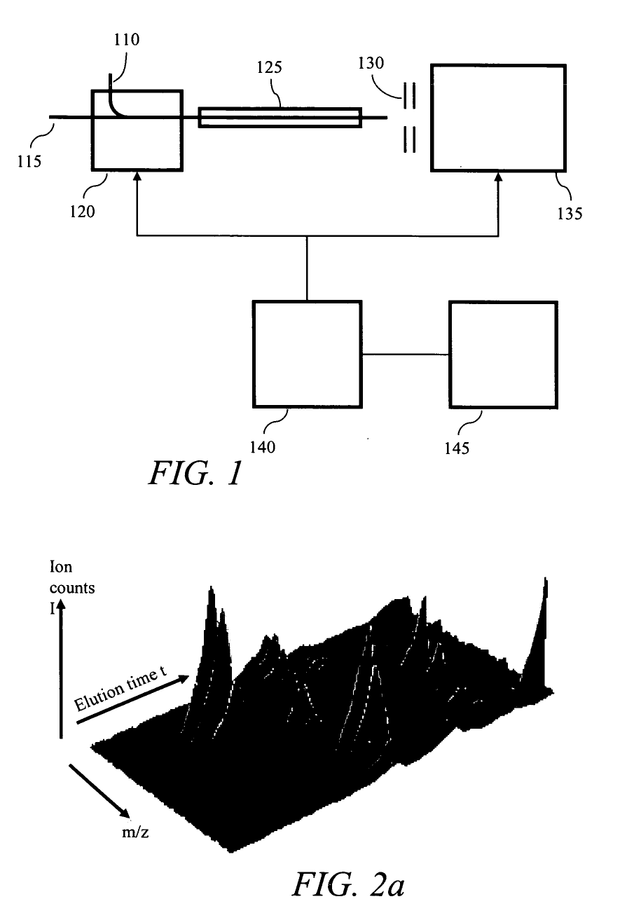

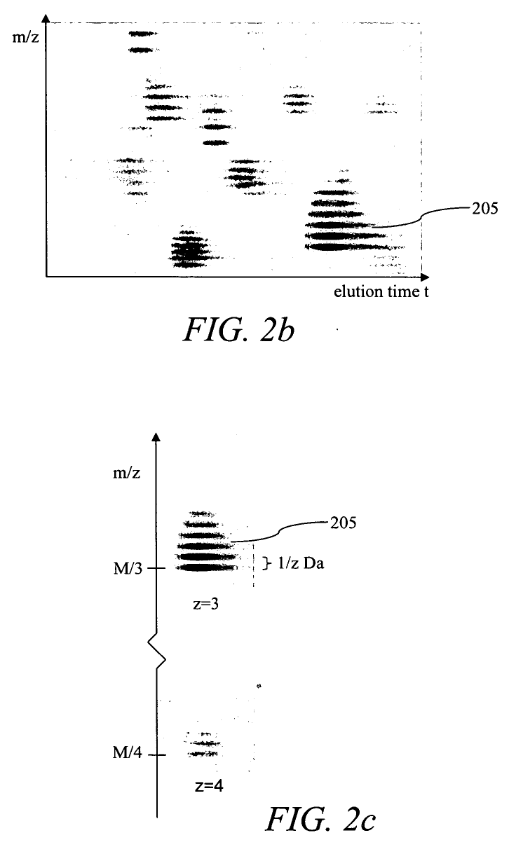

A Chromatography/Mass-Spectrometry (C/MS) analysis of a biological system is typically performed by running a plurality of samples representing different conditions in a biological system under study, through a combination of C/MS instrumentation. The chromatography can be seen as a separation method and the mass-spectrometry as a method of detection. Currently the most used and most promising method for the separation of biomolecules comprises Liquid Chromatography (LC). However, also other separation methods can be used, for example Gas Chromatography (GC). The inventive method and apparatus will be described using, but is not limited to, liquid chromatography. An instrumental setup, schematically illustrated in FIG. 1, suitable for performing LC/MS analysis according to the method of the present invention, comprises a sample inlet 110, a carrier inlet 115, a flow control unit 120, at least one chromatography column 125, a mass spectrometer interface 130, a mass spectrometer 135, a controlling means such as control unit 140 and an analyzing means such as analysis unit 145. The liquid chromatograph typically comprises a reversed phase column and is commercially available from for example LC Packings, Amsterdam, The Netherlands or Thermo Finnigan, San Jose, USA. The mass spectrometer may preferably operate according to the time of flight (TOF) or triple stage quadrupole (TSQ) principles, but other MS devices are conceivable. Commercially available spectrometers and electrospray units suitable in the measurement system according to the invention are available from for example Micromass, Manchester, UK and ThermoFinnigan, San Jose, USA. It is in the method and apparatus according to the present invention particularly favourable to use high resolution mass spectrometers but the inventive method can also be used to dramatically enhance the performance of an instrumental setup comprising a mass spectrometer of lower resolution. The controlling means 140 and analysing means 145 are typically realized by a PC or PCs with high computational and storage capacity as the computational loads will be substantial. The controlling means 140 and analysing means 145 are in communication with the chromatography column 125 and the MS 135, and possible with other units (not shown) responsible for sample preparation or transportation, for example. The method according to the invention is preferably at least partly automated and implemented as a software program or a plurality of software program modules stored and executed in the controlling means 140 and/or analysing means 145. Using the above exemplified instrumental setup, elution profiles of the type described in the background section may be produced. An example of an elution profile is depicted in FIG. 2a, having the m/z ratio represented on the y-axis, the elution time ton the x-axis, and the z-axis representing ion counts I. Each biomolecule species in the sample will typically, as will be further described below, produce characteristic variations, peaks, in the z-dimension. Due to the existence of different isotopes and different charge states, for example, each biomolecule species will typically cause a plurality of peaks.

As appreciated by the skilled in the art the instrumental setup adapted for producing elution profiles with the described characteristics, may be realized in a number of various ways, and the above should be regarded as a non limiting example of an instrumental setup adapted for performing the method according to the present invention.

In the description the use of the method and the measurement arrangement according to the present invention, will be exemplified with analysis of peptides in a biological system. The peptides are of special interest due to their importance in many biological processes. The peptides may be native or resulting from a digestion of full length protein, for example by using enzymes like trypsin. However, the method and apparatus according to the present invention are not limited to the study of peptides. A wide range of biomolecules, especially molecules with masses smaller than 10 kDa, can advantageously be analyzed with the method and apparatus disclosed herein. The term biomolecules should be interpreted as including both single biomolecules and biomolecule complexes.

A proteomic experiment typically includes a plurality of varieties e.g. a treated group and a control group of subjects, i.e. patients, animals, colonies etc., generating a large and diverse data set. The LC/MS analysis can be pictured as dispersing the signal from each peptide species in the elution time and m/z dimensions. The typically large data set and the dispersion of the signal constitutes an information handling problem. In the method according to the invention the vast amount of data is handled by alternately using refined data representations, the original elution profiles and using peptide maps generate from elution profiles. The refined data representations are for example: a consensus elution profile combining the data of several elution profiles or a differential profile highlighting differences between individual elution profiles. Throughout the method, although refined data representations are used, preferably the raw data and the links between the raw and refined data are always preserved, in order to be able to “go back” to confirm a result and to be able to perform further analysis either on the data already collected or to initiate further analysis processes. The preservation of raw data and the possibility to alternatively use refined and corresponding original raw data are useful for the checking the reliability of the results generated by a method in accordance with the present invention.

In the method according to the invention, regions of interest, corresponding to peptides showing an interesting variation over a set of samples, may be selected based on the variation behaviour, before the peptides have been identified. The concept of detecting a region with an interesting signal variation between different profiles and selecting a region of interest for further analysis, without attempting to identify the peptides before the selection, is to be regarded as part of the present invention.

As discussed above the LC/MS analysis can be pictured as a dispersion of the signal from each peptide species in the elution time and m/z dimensions and each peptide species will typically yield a plurality of peaks in the elution profile. If the resolution of the mass spectrometer is high enough different isotopes of the same peptide species will be separated in the elution profile. Characteristic “isotope ladders” 205 can be seen in the elution profiles, as exemplified in FIG. 2b. Another type of dispersion of the signal is inflicted by the experimental method. The commonly used electrospray interface of the mass spectrometer often produces several kinds of molecule-adduct ion complexes with varying number of adduct ions. These are referred to as different charge states of the peptide. As the mass spectrometer measures the mass-to-charge ratio, not just the mass, these different charge states will end up at different positions in the elution profiles. Hence one peptide species may appear in several charge states, each consisting of several molecule isotopes as illustrated in FIG. 2c. For a peptide species of mass M, containing i additional neutrons and aggregated with z adduct ions (charge state), peaks may be expected at:

$${( m / z )}_{i , z} = \frac{M + iz}{z} = ( M + i ) / z + 1 \qquad eq . 1$$

wherein it is assumed that the spacing between isotopes and the adduct ion mass are precisely 1 Da. As indicated in the figure the “distance” between different isotopes of the same peptide species will be 1/z.

If the separation of different isotopes are distinguishable or not, will depend on the mass spectrometer resolution. The resolution of the mass spectrometer may in turn depend on m/z. A peptide species will typically appear in the elution profile with separated isotopes, i.e. well defined peaks, for the charge states with low z and as less well defined “blobs” including several isotopes, for higher z.

In order to, for example, compare the abundances of certain peptide species between different samples, it is in most application advantageously to reassemble, or link, all peaks originating from the same peptide species. The aim of the reassembling is to generate a peptide map corresponding to an elution profiles. In the peptide map all dispersed signal relating to each peptide species in one elution profile is, if possible, brought together.

To be able to compare the relative abundance between different peptide species and/or the changes in abundances of certain peptides between different experimental and biological conditions, typically represented by different samples (and hence different elution profiles), it is necessary to also link peptide species across different samples represented by individual elution profiles and peptide maps thus forming a global annotation. The global annotation is preferably achieved by an automated matching process as will be described below.

Even though the theoretical relation between different peaks of the same peptide species is known according to the above, the generation of peptide maps and the matching are, in practice, not trivial tasks. The complications arise from several factors. In a typical sample a large number of different peptides are present, and peaks may be very close or overlapping, making it difficult to, taking experimental uncertainties under consideration, for example ascribe the correct charge states to a specific peptide. In addition typically not all charge states are represented and their relations are not known. Noise will always be present, both as a background noise level and as spurious noise peaks. The noise may lead to falsely identified peptides peaks. One complication of special importance is caused by experimental variations, most pronounced as an unpredictable variation in the elution time. Elution profiles from identical samples may be shifted and/or compressed or expanded in the elution time when compared to each other. The method according to the present invention offers an automated annotation process, adapted to produce a peptide map for each elution profile or from a group of elution profiles. The method produces peptide maps of high quality and reliability, and importantly, significantly reduces the time needed, in comparison with the prior art methods, for the annotation process. The method according to the present invention differentiates from the prior art methods of automated annotation in that, among other features, it is capable of reassembling isotopes as well as charge states. In addition the inventive method offers an increased effective sensitivity, as very weak signals can be detected and processed by the automated annotation. This is possible since the peak detection is performed simultaneously in both the elution time dimension and the m/z-dimension, requiring a peak to have an extension in both dimensions, giving a detection method that is less sensitive to noise.

The peptide maps produced by the annotation are the input to the matching process. The outcome of the matching, as well as the processing time needed, is highly dependent on the quality of the annotation, i.e. the peptide maps. The automated annotation method according to the present invention, which gives accurate and reliable peptide maps, is required for an effective and accurate matching process, and hence to achieve a correct global annotation. The global annotation is in turn needed for a reliable statistical evaluation of the experiment.

Different type of chemical pre-processing of the samples can also cause differences in the mass of the biomolecule and hence a splitting of the signal. Even if the differences are wanted and aimed to facilitate a certain analysis, the effect of the differences must be accounted for in any reassembling of the biomolecule peaks in the elution profiles. The method of automated annotation according to the present invention is easily adapted also for this type of wanted mass differences.

In a plurality of the analysing steps of the method according to the invention the analysis is performed in the two-dimensional space defined by the elution time and the m/z. This might at first sight seem like a complication, but will be shown to simplify the process of re-assembling the spread out signal from each peptide, for example. The concept of simultaneously using both the elution time dimension and the m/z dimension of an elution profile is advantageous

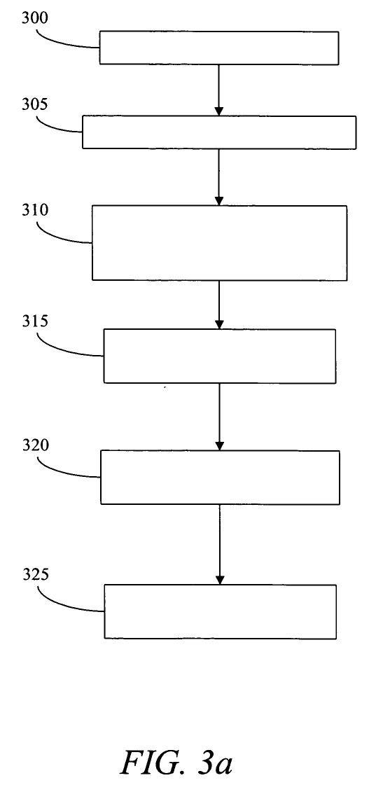

The main steps of method according to the present invention, which will be described with references to the flowchart of FIG. 3, comprises the steps of:
performing 300 a C/MS analysis.
generating 305 first elution profiles.
reassembling dispersed signals and generating peptide maps 310 by an automated annotation process. In the peptide map all dispersed signals relating to each peptide species in one elution profile are, if possible, brought together, e.g. the different charge states and isotopes of a peptide species are reassembled. The automated annotating is simultaneously based on both the elution time-dimension and the m/z-dimension.
matching 315 the individual peptide maps to each other. The matching links the peptide species across the different samples, for example representing different experimental and biological conditions, and gives a global annotation.
performing measurement and evaluation 320 for profiling the relative abundance of some of the individual peptide species across different experimental and biological conditions. The abundance profiles are based on the global annotation obtained in the preceding steps.
optionally defining subsets 325 of peptide species for further analysis. The subset defines “peptides of interest” for further characterisation and possibly identification, using MS/MS, for example. The subsets can be defined automatically or manually.

The steps of the method will be described in detail below:

Performing 300 an C/MS Analysis and Generating First Elution Profiles 305

Two or more biomolecule containing samples are run through a combination of LC/MS instrumentation according to the setup described above. The samples could typically represent different conditions in a biological system being studied. The simplest case is a differential experiment aiming at highlighting biomolecule species for which there is a large change in abundance between two different experimental conditions. A more advanced experimental design involves more than two conditions and/or introduces replication, i.e., the use of more than one sample per experimental condition. By the use of well-established statistical methods it is possible to assign statistical significance to abundance changes between the different conditions.

The measurement system according to FIG. 1 is used for carrying out the method according to the invention. Each run resulted in an elution profile. Each individual data point in the elution profile represented an intensity value, or ion count, obtained from the MS detector for a particular chromatographical elution time and a particular m/z value. 3D representations of these elution profiles were drawn in which the y-axis showed the m/z ratio, the x-axis showed the elution time and the z-axis represented ion counts. In certain cases, depending on the characteristics of the measurement system, a re-sampling is needed to compensate for differences in the sampling in the m/z-dimension. This is an established and well-known procedure. The step of generating typically produces a set of first elution profiles in which a characteristic variation in the signal intensity is an indication of the existence of a, or part of a, specific peptide species.

Generating Peptide Maps 310 by an Automated Annotation Process

The automated annotation process, according to the method of the present invention, automatically reassembles signals originating from the same peptide species dispersed in the elution profile and appearing as a plurality of peaks. The peaks typically range from well-defined to weak and diffuse for the same peptide species. The automated annotation process generates a peptide map for each elution profile.

The automated annotation algorithm starts by detecting primary features presumably corresponding to peaks in the signal variation of the elution profile. Primary features may comprise e.g. local maxima in the signal intensity, seeds from thresholding morphological operations or positions selected by analysis of gradients. Spots are compact areas of high intensity, which are detected starting from the primary features. Spots may correspond to individual isotopic peaks, or to isotopic peak clusters when the instrument resolution is not good enough to separate them. Spots may also originate from noise and data acquisition artefacts. The primary feature detection and spot detection steps make use of the local surroundings of the data points in both the m/z and elution time dimensions. A spot must have at least a predefined extension in both dimensions. In that way noise peaks, for example, are avoided.

When a spot is found, attempts are made to put it into context, i.e., to find additional traces of the peptide species that gave rise to the spot in the elution profile. As previously described, these traces are highly structured; the spot corresponds to a certain charge state and possibly a certain molecule isotope of the peptide species, and there may also be spots for other molecule isotopes and additional charge states. If a labelling method is used, there may also be spots corresponding to differently labelled versions of the same peptide species. Thus, a peptide map entry for the peptide species is constructed, starting from a single spot. This step is carried out for each spot.

The last step in the process is a refinement step, where duplicate entries are removed and overlaps are resolved. A peptide species may be detected several times by the algorithm (e.g. once for each charge state), which leads to duplicate entries in the peptide map. Such duplication is detected by systematic comparison and duplicate entries are removed either automatically or manually. There may also be regions where two or more peptide species overlap, due to insufficient chromatographic separation. A region where there is a large overlap between two peptide species cannot be used for measurements of the amounts of either species, and may therefore have to be removed from the map entries of both species or otherwise be indicated as being unreliable.

Referring now to the flowchart of FIG. 3a, the step of automated annotation 310 may according to the above description comprise the following substeps, described with reference to the flowchart of FIG. 3b:
310:1 finding and marking primary features corresponding to peaks in the signal variation of the first elution profile,
310:2 defining a first set of spots, each spot comprising at least one primary feature. The spots will have a variable extension in the m/z-dimension and a variable extension in the elution time dimension. A spot is assumed to correspond to a specific charge state and an isotope or group of isotopes of a biomolecule, and possibly to a specific chemical label;
310:3 constructing a peptide map entry for each spot, i.e., detecting a set of regions, with a known structural relationship, confining the patterns from one peptide species within the elution profile. Depending on the C-MS instrumentation and labelling scheme used, this step comprises one or more of the substeps 310:3a-c. If the findings for a particular charge z are significant and consistent, they are used to create a peptide map entry. If no suitable charge can be found, an incomplete peptide map entry is created from the spot itself.
310:3:1 for each putative charge z, detect additional isotopes at m/z±1/z, m/z±2/z, etc., if possible.
310:3:2 for each putative charge z, detect additional charge states at (m−1)/(z±1)+1, (m−1)/(z±2)+1, etc., if possible.
310:3:3 for each putative charge z, detect different label variants. The expected displacement in m/z and elution time depends on the specific labelling scheme used.
310:4 Optionally duplicate peptide map entries are removed.
310:5 Optionally overlapping peptide map entries are adjusted or indicated as being unreliable.
310:6 Optionally manual verification of the resulting peptide map.

In order to assess the significance and consistence of the detected isotopes, charge states, and label varieties of step 310:3, a number of measures can be used, e.g.:
a) similarity with respect to the signal pattern over elution time between the detected feature and the spot. This can be pictured as, for example, that the shape of a peak corresponding to one charge state or isotope of a biomolecule species is likely to resemble the shape of another peak corresponding to another charge state or isotope, respectively of the same biomolecule species. A high degree of resemblance indicates a high probability that the detected feature and the spot originate from the same peptide species;
b) in the case of charge states: similarity in isotope distribution between charge states. The different charge states of the same biomolecule species can be expected to show similar isotope distribution. Therefore, if the “isotope ladders” of the different charge states shows a high resemblance it is probable that the detected feature and the spot correspond to the same biomolecule species;
c) signal-to-noise ratio;
d) signal intensity;
e) in the case of isotopes: similarity to a predetermined model isotope distribution giving an indication on how probable an assumed isotope is for the given peptide mass. Predetermined model isotope distribution and methods of obtaining such are given in “Determination of Monoisotopic Masses and Ion Populations for Large Biomolecules from resolved Isotopic Distributions” by Senko et al, J Am Soc Mass Spectrum, 1995, 6, 229-233.

As can be seen, these measures make extensive use of both the m/z and elution time dimensions. The measures a) and b) are examples of how the method according to the invention uses a priori knowledge of the structure of the dispersion of the signal to verify an assumption on charge state and isotope, for example. The above measure can preferably be combined.

If the different isotopes of a peptide species are distinguishable or not, will depend on the charge state z, and the mass spectrometer resolution at the particular m/z ratio. A peptide species will typically appear in the elution profile with separated isotopes, i.e. well-defined peaks, for the charge states with low z and as less well defined “blobs” including several isotopes, for higher z. In the case where a mass spectrometer operating according to the time-of-flight (TOF) principle is used, the mass spectrometer resolution also depends on m/z, imposing a complication in the isotope detection step 310:3:1.

In one embodiment of the present invention peptide map entry construction step 310:3 is improved by including different modes reflecting the resolution characteristics of the mass spectrometer. The resolution of the spectrometer is typically assumed to be dependent on m/z and described by a spectrometer resolution function R(m/z), as stated by the mass spectrometer manufacturer. The peptide map entry construction step 310:3 may then operate in at least two different modes: a high resolution mode and a low resolution mode, wherein the shifting between the modes is dynamic. The criteria for shifting between the modes are for example dependent on R(m/z) and z. In this embodiment, using the two resolution modes and the dynamic switching between them, the algorithm will only search for different isotopes of a peptide species for charge states where isotope resolution is expected according to the mass spectrometer resolution. This not only saves processing time, it also improves the quality and reliability of the produced peptide maps. This in turn is a prerequisite for a reliable result of the subsequent matching step 315.

In the case where the resolution of the spectrometer is well-described by the function R(m/z), an effective resolution βR can be used for setting up a criteria for shifting between the resolution modes. β is an empirically predefined parameter relating to a required minimum difference between peaks and valleys in the elution profiles. A suitable value of β is 0.85 (unitless). R(m/z) depends on the properties of the mass spectrometer and is usually available from the manufacturer. For a given m/z and z the high resolution mode is used if:

$$\frac{m}{z} < \frac{1}{z} β R \qquad eq . 3$$

and the low resolution mode is used otherwise.

A background noise will always be present in the elution profiles, and the annotation process may be preceded by a noise removing step. All signal intensity below a threshold may be removed, for example. Since the signal level may fluctuate significantly between elution profiles, any signal intensity thresholds should preferably be chosen individually for each elution profile. Suitable background and peak thresholds are taken to be the 95^{th} and 99^{th} percentiles of the intensity distribution of the elution profile, respectively.

A detailed example of an automated annotation algorithm, representing a current best mode of operation, is presented under the section Implementation examples.

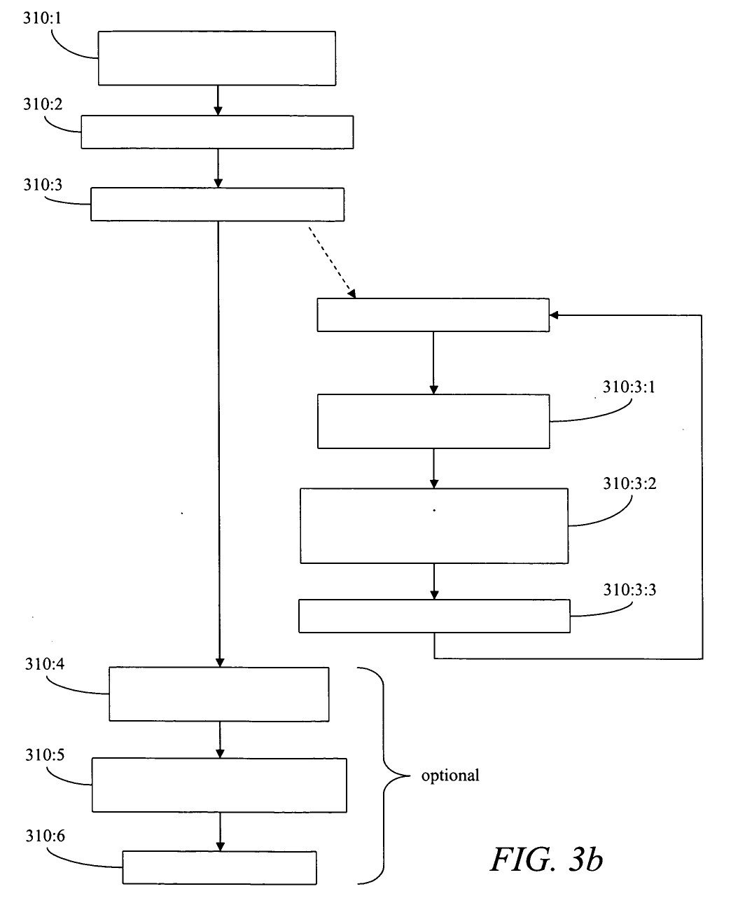

The usefulness of the method according to the present invention, compared to some prior art methods, is illustrated in FIG. 4. In the schematic figure two isotope peaks A₁ and A₂ of a peptide A is partly interleaved with two isotope peaks B₁ and B₂ of a peptide B. The prior art methods, for example the methods referred to in the background section, analysing one or a few MS-spectra at the time, and typically not all available spectra, are likely to interpret the data as three different peptides (the spectra chosen along lines e, f and g, for example). The method according to the invention, simultaneously considering both the retention time dimension and the m/z dimension will correctly identify two peptides with two isotope peaks each.

Matching Peptide Maps 315

The aim of the matching step 330 is to generate the global annotation which is needed for the abundance profiles for individual peptides across different samples. The matching links the peptide species across the different elution profiles, for example representing different experimental and biological conditions.

In certain application the number of biomolecules in one map will not be very large (typically on the order of 100-10,000) and the mass spectrometer can give a very accurate and specific mass measurement for each peptide. In these cases, and since the elution profiles are aligned, the matching of the peptide maps will be a simple projection of the peptide map of one elution profile (or consensus) onto another elution profile.

In other cases the unique masses of individual peptides can not be fully resolved and clusters will be formed. These clusters must be resolved in order to get the global annotation. This is preferably achieved by treating the matching process as an optimization problem. Those skilled in the art will appreciate that many different optimization methods may be used for this type of problem, including greedy algorithms, simulated annealing, dynamic programming or genetic algorithms.

An example of a matching algorithm, suitable to be combined with the automated annotation, which has generated peptide maps, is given under the section Implementation Examples.

Abundance Measurement 320

For each elution profile with an associated peptide map, the signal intensity over the data points belonging to each peptide species in the map can be integrated. This yields an intensity measurement for each peptide species, and (optionally) for its charge states and molecule isotopes.

A data point in an elution profile is a measurement of the number of ions that were detected in a certain mass-to-charge ratio interval, during a certain time interval. Provided that the ions all come from the same peptide species, this can be can regarded as a measurement of the amount of the species in the sample. Measurements cannot be compared directly between species, because different molecule species are ionised to different extent in the mass spectrometer. However, the previously mentioned investigation by Sköld et al indicates that the measurements are at least repeatable. Since the peptide species are matched the relative abundance of peptide species between the different samples can be established.

Certain measures can be taken to further increase the accuracy of the abundance and relative abundance: A normalisation procedure can be applied to e.g. compensate uneven sample loadings among the LC/MS runs; and internal standards (spikes), i.e. known amounts of certain peptide species can be added to the samples before the LC/MS analysis. In each experiment there will be a large number of elution profiles, yielding a large number of abundance measurements. These measurements have a high degree of structure. There is the peptide species—charge state—isotope relation, to begin with, which may be aggregated to reduce the number of measurements. There is also the experimental design that relates the runs to each other and adds a number of factors/dimensions to the data set. In many cases further analysis of the data will facilitate the interpretation. This kind of data is preferentially analysed by multivariate statistical methods for example ANOVA (Analysis of Variance), PCA (Principal Components Analysis) and FA (Factor Analysis). Various regression methods can also prove useful for model building. The analysis may be performed using dedicated, custom-built software, or by general-purpose statistical and data analysis packages such as SAS (SAS Institute Inc, Cary, N.C. USA) or Spotfire (Spotfire, U.S. Headquarters, Somerville, Mass., USA).

Defining Subsets of Peptide Species for Further Analysis 325

One aim of the method according to the present invention is to be able to define a subset of peptide species for further analysis from the samples, represented by the peptide maps. The preceding steps of the method have made it possible to select peptides of interest since their abundance and/or relative abundance across different samples is measured. The subset of peptide species may be peptides that show a high variation in abundance between samples, or show a statistically significant variation between replica groups of samples, or yield individual measurements with high abundances. The selection of these biomolecules may be achieved automatically, by applying user-specified thresholds for the selection criteria. Selection criteria are for example “all peptides with significant variation between samples above a threshold”, “the ten peptides with the highest abundance” etc. The selection may also be done manually, or by a combination of manual and automated selection. The selection process, manual or automated, may advantageously use a differential profile to highlight the differences between samples.

The further analysis of the subsets of peptides typically and preferably comprises identification or further characterisation by MS/MS. The previous exemplified, in connection to FIG. 1, commercially available measurement systems are capable of also performing MS/MS analysis.

In a further embodiment of the invention a first portion of a sample is analysed according to the above method and at least one subset of peptide species is selected. The elapsed time when they are supposed to elute, and what is supposed to elute in-between are known from the representation of the elution profile, and therefore it is possible to construct a list of features to be on the lookout for during an upcoming identification/characterisation run on a second portion of the same sample. These features consist of the identification candidates themselves, taken together with a number of “sentinel features” that act as markers/milestones that enables corrections to be made for experimental variation in elution time. The subset is then further analysed with MS/MS. By using the list of features the elaborate MS/MS analysis is essentially only performed on the selected peptides. The ability to construct this list is provided by the method according to the invention by the raw data (elution profiles) and the links between the global annotation, the peptide maps and the raw data being preserved.

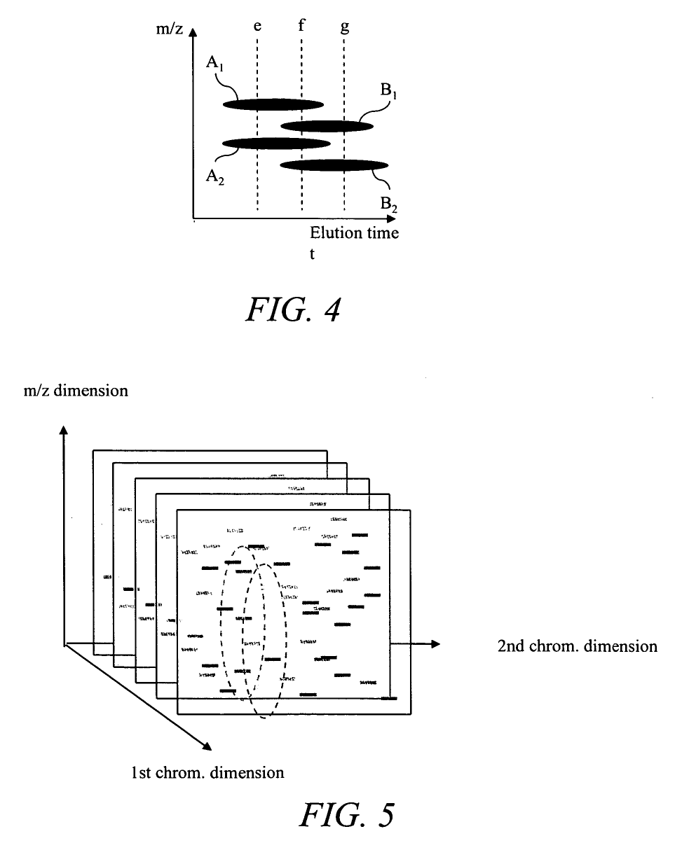

In the area of chromatography much attention has lately been given to the possibilities of introducing more than one separating step. These techniques are referred to as multidimensional chromatography and are well known in the art. Multidimensional liquid chromatography is advantageously combined with mass spectrometry (MDLC/MS). By introducing additional separation steps more complex samples, for example blood plasma, may be purposefully analysed. A 2-dimensional expansion of the measurement system described with reference to FIG. 1. could include a further chromatography column, giving a system with for example an ion exchange column (IEX) followed by a reversed phase column (RPC) combined with one of the above exemplified mass-spectrometers as the detector. In FIG. 5 is the output of such a 2DLC/MS measurement system is schematically depicted. Compared to the previously described 1D LC/MS the result can be seen as a further chromatographic dimension has been added. Each sample will in the 2DLC/MS give raise to a plurality of elution profiles corresponding to the additional separation afforded by the added chromatography column.

The method according to the present invention of automatically annotating elution profiles will work also for this type of experiment, without any non-trivial adaptations. The elution profiles from a MDLC/MS are annotated in the same manner as in the described 1DLC/MS. Since the multidimensionality multiplies the number of elution profile, and the amount of data will be very large also in an experiment involving a rather small number of samples, the method according to the invention will be particularly useful.

Additionally, other types of multidimensionality, created by additional separation steps e.g. electrophoresis and iso-electric focusing (IES) or other methods, may in the same manner be handled by the method according to the present invention.

Methods of chemically labelling molecules in samples have received an increasing attention. The idea of chemical labelling is to treat samples from, for example, a treated group and a control group, exactly the same way through the sampling, preparation and measurement procedures. The chemical labels are used to separate the groups at a late stage in the analysis. A chemical labelling of particular interest in the area of proteomics and LC/MS-techniques is mass labelling.

The method of automated annotation according to the present invention handles chemical labels, for example mass labels, as described in the step 310:3:3. As appreciated by those skilled in the art, other types of labels, including, for example, isotope labels, may be used in the same manner.

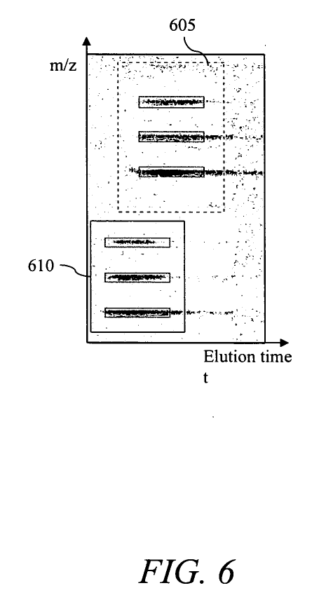

Illustrated in FIG. 6 is an elution profile showing two regions 605 (dashed) and 610 (solid) originating from a peptide that has been given different mass labels. The method according to the invention with the above modification identifies the two regions as originating from the same peptide species given different mass labels.

An example of an experiment utilising mass labels and the method of automated annotation according to the present invention is given under the section Implementation Examples.

In the automated alignment of the present invention, and also in the annotation and matching process, the original data of the elution profiles is preferably preserved as well as the correlations between refined data and the original data. In addition the method is very visual, and preferably visualized with the aid of computer graphics, for example how peptide maps are projected onto elution profiles. This gives an ability to visualise the steps of the method as well as confirm and verify a high level result with original data. For example to check the consistence of a global annotation with the first elution profiles. This is of special importance if, for example, advanced statistical methods are needed for the abundance measurement. Such advanced methods, however powerful, may in certain cases produce doubtful results even if the statistical measure may indicate a high accuracy. In these cases, the ability to trace the result back to original data and the visual nature of the results and interim results such as elution profiles and peptide maps are of high value.

### EXAMPLES

Below, the present invention will be explained in more detail by way of examples, which however are not to be construed as limiting the present invention as defined by the appended claims. All references given below and elsewhere in the present specification are hereby included herein by reference.

A. Auto-Annotating an LC/MS Elution Profile

1. Spot Detection

1.1. Selection of Background and Peak Thresholds

Because the signal level may fluctuate significantly between elution profiles, any signal intensity thresholds should be chosen individually for each elution profile. In this implementation, the background and peak thresholds are taken to be the 95^{th} and 99^{th} percentiles of the intensity distribution of the elution profile, respectively.

1.2. Detection of Primary Features

Each data point in the elution profile is compared with its neighbours in order to find local maxima. Any local maxima above the peak threshold are considered valid primary features.

1.3. Spot Detection (Corresponds to 310:2)

For each local maximum, a m/z interval centered at the maximum is set up. The width of the interval is taken to be the FWHM (full width at half maximum) for a mass spectrometer peak at that particular m/z, a figure which is available from the manufacturer of the mass spectrometer.

An elution time interval is then found by scanning for signal above the background threshold within the m/z interval in both directions along the elution time axis. A spot is formed by combining the m/z interval with the elution time interval.

A thresholding procedure is applied to remove spots that have a too short time extent, assuming that they result from spurious noise.

2. Peptide Map Entry Construction (Peptide Pattern Reassembly) (Corresponds to 310:3)

This step is carried out for each spot individually. Spots are ordered with respect to decreasing peak intensity.

2.1. Seed-Spot Charge Screening

The set of putative charges z is screened for candidates in steps 2.1.1-2.1.3. Each z that passes the screening is assigned a score, and the z with the best score is selected.

First, try to detect isotopes, if a) meaningful (mass spec resolution dependent) and b) non-blob.

Then, try the charge states,

Then, try the labels, if a labelling scheme is used.

Finally, after selecting one z, do some refinement.

2.1.1. Isotope Detection

if

$$\frac{m}{z} < \frac{1}{z} β R$$

(i.e. high-res mode is suitable) and the peak is well-resolved (test by comparing to a model peak), then
search for isotopes with spacing 1/z Da. The minimum number of detectable isotopes is estimated from the average isotope distribution (the averaging of a certain mass is an average of all peptides of that mass). The tentative isotope positions m/z±1/z, ±2/z, are investigated:
the signal must be above the background threshold
the signal must be well-behaved between the isotope positions (filtering out peaks from higher z's)

If there are enough valid isotope positions, the charge state passes the screening.

2.1.2 Neighbour Charge State Detection

detect additional charge states at (m−1)/(z±1)+1

using the same time interval as the spot, look for

signal above background
similarity to the spot signal pattern
   small mass deviations are allowed so that an incorrect calibration of the mass spectrometer does not ruin the results.
   2.1.3 Detection of Other Labels

This has to be specifically implemented for each labelling scheme.

2.1.4. Peptide Map Entry Refinement

detect more charge states using basically the same method as above;
refine time intervals, isotope intervals, and so on.
isotope shift:

It is possible (even likely for large peptides) that the lowest isotope has very low abundance and therefore won't be detected. The empirical isotope distribution is matched to various shifted versions of the average isotope distribution, and the closest match is selected for the calculation of the peptide mass.

A subsequent step is to find the start of the isotope ladder that contains the spot. This is necessary for assigning the correct mass to the peptide species. Simply taking the first detectable spot to be the start does not work for large peptides or proteins, where the relative abundance of the first molecule isotope is almost zero. Instead, an approximate molecule isotope distribution is calculated as described by Senko et al, which is then fit to the region surrounding the spot for a number of possible integer-mass shifts.

3. Peptide Map Refinement (Corresponds to 310:4-5)

In this step, overlapping peptides are detected and the overlaps resolved. The method identifies four cases and handles them separately:
large overlap, same z;
large overlap, different z's, both corresponding to seed charge states;
same mass (long peaks/split peaks);
other kinds of overlap, excluding very small overlaps.
   B. Matching Algorithm

The algorithm takes two or more peptide maps as input. The output is a match table, holding one column for each peptide map. The rows of the table correspond to unique peptides. Non-empty table cells represent a mapping from a unique peptide (table row) to a peptide in a particular map (table column). An empty table cell indicates that a unique peptide does not match any peptide in a particular peptide map. For each peptide in each map, the mass (M/z and usually M) and the elution time are known.

The matching is performed in two steps. Both steps employ a greedy algorithm. A greedy algorithm is not optimal, but scales well with problem size and therefore selected. Other algorithms such as simulated annealing or genetic algorithms could also be employed.

1. Cluster Formation:

A cluster is a putative row in the match table. In the first step, the optimal cluster for each peptide is found, at this stage ignoring conflicts with other clusters.

All peptide maps are joined to form a large peptide list. The list is sorted with respect to M (or M/z if charges are not available). For each entry in the list, the optimal cluster is identified by exhaustive search (within a mass tolerance). The optimal cluster for a given list entry (i.e., peptide) is defined as the best-scoring cluster that contains that particular list entry (called the reference) and at most one list entry from all other maps, fulfilling the requirements: a) the mass difference between the peptide and the reference must be within a predefined limit, and b) the peptide does not belong to a selected cluster (see below).

Each cluster is assigned a score, which is calculated as the sum of all pairwise elution time difference scores within the cluster:

$$s = {\sum }_{i} {\sum }_{j > i} min \{ 1 , \frac{τ}{\vert {t}_{i} - {t}_{j} \vert} \} \qquad eq . 4$$

wherein |tᵢ−tⱼ| is the pairwise elution time difference. The parameter τ is interpreted as the largest time difference that is considered a perfect match. Score 1 is considered a perfect match between two peptides, and 0 an infinitely bad match. A cluster must not contain a pair with zero score.
2. Selection of Clusters:

In the second step the clusters are sorted with respect to score. The following procedure is then iterated as long as there are any clusters left:

a) the best-scoring cluster is found using linear search.

b) the cluster formation algorithm, 1) is run on that cluster again. If the score has decreased, it is assume that some of the peptides in the cluster now belong to a selected cluster; the cluster score is updated and the procedure restarts. It may also happen that the score increases; this is due to the non-optimality of the greedy algorithm and is ignored.

c) the best-scoring cluster is selected, i.e., copied to the match table.

This exemplary algorithm may preferably be extended in several ways. For example with a limitation on how well the elution times must match in order to make a valid match. A simple way of solving this problem is to append a cutoff threshold to the cluster formation requirements. Alternatively dynamic thresholds, for example based on a statistical measure on how well all peptides match can be used.

C. Annotation of a Mass Labelled Sample

Consider a simple experiment with the intent to examine of the effects of a drug. There are two experimental varieties; a “treated” variety that receives a drug treatment, and a “control” variety that is treated identically except that the drug is replaced by placebo.

1: Collect tissue samples from animals of each variety and prepare them for LC-MS analysis.

2: Label each sample with a different label. In the case of ICAT, the labels are molecules that bind to the cysteine residues in the peptides. One label contains eight hydrogen atoms, and the other kind contains eight deuterium atoms.

3: Pool the labelled samples.

4: Purify the labelled peptides on an affinity column. Peptides and other molecules that lack a label flow right through and are removed, leading to less background in the subsequent analysis steps.

5: Perform an LC-MS analysis of the purified, pooled sample. In this example, peptides will show up in pairs separated by eight Da.

6: Annotate the profile, i.e., run the peptide detection algorithm, and quantitate each peptide.

7: Identify peptide pairs (or n-tuples if there are more than two labels) and mark each labelled peptide with its corresponding variety—this is easily done because the labelling scheme (and therefore the expected mass difference) is known, and the mass difference should not lead to large differences in elution time. The outcome of this process is a cross-table of <mass, control-intensity, treated-intensity> entries that can be further analysed by appropriate statistical methods. To be performed in step 310:3:3 of the annotation algorithm.

It is apparent that many modifications and variations of the invention as hereinabove set forth may be made without departing from the spirit and scope thereof. The specific embodiments described are given by way of example only, and the invention is limited only by the terms of the appended claims.

1. A method of performing a combined Chromatography and Mass Spectrometry analysis (C/MS) on at least one sample for the characterization of biomolecules species in the sample, which method comprises the steps of:
performing an C/MS analysis (300);
generating at least one first elution profile (305), said first elution profile being a multidimensional representation of the data resulting from the C/MS analysis wherein one dimension is an elution time of the chromatography, and one dimension is mass to charge ratio (m/z), and at least one dimension is a signal intensity, and in which of the elution profile a characteristic any variation in the signal intensity is an indication of the existence of a specific biomolecule species, and wherein the signal from each biomolecule species is dispersed forming a plurality of signal peaks associated with each biomolecule species in the elution profile; and
reassembling the dispersed signal originating from one biomolecule species in the elution profile (310);
and further wherein said reassembling step includes an automated annotation adapted to reassemble signal variations in the elution profile that originate from the same biomolecule species and generating a biomolecule map, said automated annotating being simultaneously based on at least both the elution time-dimension and the m/z-dimension.

2. The C/MS analysis method of claim 1, wherein the dispersion of signal from each biomolecule species arises from different isotopes and/or charge states of the biomolecule species, and wherein the automated annotating reassembles the signal dispersion for essentially each biomolecule caused by the different isotopes and/or different charge states of the biomolecule species.

3. The C/MS analysis method of claim 2, wherein the sample that comprises biomolecules species includes different chemical labels, resulting in, at least, a first chemically labelled biomolecule with a first label and a second mass-labelled biomolecule with a second label, said labels causing a further dispersion of the signal in the elution profile, and wherein the automated annotation reassembles the signal dispersion caused by the chemical labelling.

4. The C/MS analysis method of claim 3, wherein the chemical labels are mass labels resulting in at least a first mass labelled biomolecule with a first mass label and a second mass labelled biomolecule with a second mass label, and wherein the mass difference causes a dispersion of the signal in the elution profiles, and wherein the automated annotation reassembles the signal dispersion inflicted by the mass labelling.

5. The C/MS analysis method of claim 3, wherein the chemical labels are isotope labels resulting in at least a first isotope labelled biomolecule including isotopes of a first type and a second isotope labelled biomolecule including isotopes of a second type, the different isotopes causing a dispersion of the signal in the elution profiles, and wherein the automated annotation reassembles the signal dispersion inflicted by the isotope labelling.

6. The C/MS analysis method of claim 2, wherein the automated annotation in the reassembling of dispersed signals uses knowledge of the mass spectrometer resolution.

7. The C/MS analysis method of claim 6, wherein the automated annotation in the reassembling of dispersed signals uses a priori assumptions on the relations between different charge states and/or different isotopes of the same biomolecule species in the reassembling of dispersed signals.

8. The C/MS analysis method of claim 6, wherein the automated annotation in the reassembling of dispersed signals uses an assumption that a first signal pattern associated with a first charge state of a biomolecule species has an resemblance with a second signal pattern associated with a second charge state of the biomolecule species.

9. The C/MS analysis method of claim 6, wherein the automated annotation in the reassembling of dispersed signals uses an assumption that a first isotope distribution associated with a first charge state of a biomolecule species has an resemblance with a second isotope distribution associated with a second charge state of the biomolecule species.

10. The C/MS analysis method of claim 6, wherein the automated annotating comprises the steps of:
a) finding and marking peaks in the signal variation of the first elution profile (310:1);
b) defining a first set of spots, wherein each spot including at least one primary feature and said spots have a variable extension in the m/z-dimension and a variable extension in the elution time dimension, and each spot is assumed to correspond at least to a specific charge state and an isotope or group of isotopes of a biomolecule (310:2);
c) constructing a peptide map entry for each spot by detecting a set of regions with a known structural relationship and confining the patterns from one peptide species within the elution profile (310:3); and
d) repeating steps (b) to (d) for each spot, and wherein,
in the step of constructing a peptide map entry is created if for the charge state the structural relationships of the set of regions are essentially consistent and significant, and if no charge state giving essentially known the structural relationships of the set of regions can be found, an incomplete peptide map entry is created from the spot itself.

11. The C/MS analysis method of claim 10, wherein, the step of constructing a peptide entry comprises, for each putative charge z, detecting additional isotopes at m/z±1/z, m/z±2/z, etc. (310:3:1).

12. The C/MS analysis method of claim 10, wherein, the step of constructing a peptide entry comprises, for each putative charge z, detecting additional charge states at (m−1)/(z±1)+1, (m−1)/(z±2)+1, etc. (310:3:2).

13. The C/MS analysis method of claim 10 wherein, the step of constructing a peptide entry comprises, for each putative charge z, detecting different label variants, wherein the expected displacement is specific for the labelling scheme used (310:3:3).

14. The C/MS analysis method of claim 10, wherein the step of constructing a peptide entry of the automated annotating comprises at least two different modes reflecting the resolution characteristics of the mass spectrometer.

15. The C/MS analysis method of claim 14, wherein the resolution of the spectrometer is dependent on m/z and described by a spectrometer resolution parameter (R(m/z)), and the charge state assignment step includes a high resolution mode and a low resolution mode, wherein the shifting between the modes is dynamical, and the criteria for shifting between the modes is dependent on m/z, z and the spectrometer resolution parameter.

16. The C/MS analysis method of claim 15, wherein for given m/z and z values the high resolution mode is used if:
$$\frac{m}{z} < \frac{1}{z} β R$$
wherein R spectrometer resolution parameter and β is an empirically predefined parameter, and
the low resolution mode is used otherwise.

17. The C/MS analysis method of claim 1, wherein the method further comprises the steps of:
matching individual biomolecule maps (315) generated in the reassembling step (310), to form a global annotation;
performing measurement and evaluation (320) for profiling a relative abundance of some of the individual biomolecule species across different samples, wherein the abundance profiles are based on the global annotation obtained in the preceding steps.

18. The C/MS analysis method of claim 17, wherein the method further comprises a step of defining subsets of biomolecule species (325), said step of defining subsets adapted for selecting subsets for further analysis, and the selection being based on variations in a relative abundance of some biomolecule species across different samples.

19. A measurement system for performing a combined Chromatography and Mass Spectrometry analysis (C/MS) on at least one sample for characterization of biomolecules species in the sample, wherein the measurement system comprises at least one chromatography column (125), a mass spectrometer interface (130), a mass spectrometer (135) and means for control and analysis (140,145), and the measurement system is adapted to:
perform an C/MS analysis;
generate at least one first elution profile, said first elution profile being a multidimensional representations of the data resulting from the C/MS analysis wherein one dimension is an elution time of the chromatography, and one dimension is mass to charge ratio (m/z), and at least one dimension a signal intensity, and in which of the elution profile a characteristic any variation in the signal intensity is an indication of the existence of a specific biomolecule species, and wherein the signal from each biomolecule species is dispersed forming a plurality of signal peaks associated with each biomolecule species in the elution profile; and
reassemble the dispersed signal originating from one biomolecule species in the elution profile,
and further wherein said measurement system is that reassembling methodology includes an automated annotation adapted to reassemble signal variations in the elution profile that originate from the same biomolecule species and generating a biomolecule map, said automated annotating being simultaneously based on at least both the elution time-dimension and the m/z-dimension.

20. The measurement system of claim 19, further adapted to, in the automated annotation of dispersed signals, use knowledge of the mass spectrometer resolution for reassembling of signals originating from the same biomolecule species.

21. The measurement system of claim 20, wherein the automated annotating may operate in at least two different modes reflecting the resolution characteristics of the mass spectrometer.

22. The measurement system of claim 21, wherein resolution of the spectrometer is dependent on m/z and described by a spectrometer resolution parameter (R(m/z)), and the charge state assignment step includes a high resolution mode and a low resolution mode, wherein the shifting between the modes is dynamical, and the criteria for shifting between the modes is dependent on m/z, z and the spectrometer resolution parameter.

23. Computer program products directly loadable into the internal memory of a processing means within the means for controlling an analysing (140,145), comprising the software code means adapted for controlling the steps of claim 1.

24. Computer program products stored on a computer usable medium, comprising readable program adapted for causing a processing means in a processing unit within the means for controlling an analysing (140,145), to control an execution of the steps of claim 1.
